(19) United States
(12) Patent Application Publication
(10) Pub. No.: US 2011/0313412 A1
(43) Pub. Date: Dec. 22, 2011

(54) TISSUE INTERFACE SYSTEM AND METHOD

(75) Inventors: Steven Kim, Los Altos, CA (US); Daniel Francis, Mountain View, CA (US); Jessi E. Johnson, Sunnyvale, CA (US); Alexey Salamini, San Francisco, CA (US); Ted Su, Sunnyvale, CA (US); Dong Hoon Chun, Sunnyvale, CA (US); Yoav Ben-Haim, San Francisco, CA (US); Christopher Loew, Palo Alto, CA (US)

(73) Assignee: MIRAMAR LABS, INC., Sunnyvale, CA (US)

(21) Appl. No.: 13/201,819

(22) PCT Filed: Feb. 23, 2010
(86) PCT No.: PCT/US2010/025124

§ 371 Date: Sep. 2, 2011

Related U.S. Application Data
(60) Provisional application No. 61/208,315, filed on Feb. 23, 2009.
(60) Provisional application No. 61/279,153, filed on Oct. 16, 2009.

(30) Foreign Application Priority Data
Apr. 17, 2009 (US) PCT/US2009/002403
Oct. 22, 2009 (US) PCT/US2009/005772

(51) Int. Cl. A61B 18/18

(52) U.S. Cl. 606/33

(57) ABSTRACT

An applicator-tissue interface is disclosed for use in connection with medical device treatment applicators. The interface provides a cover to protect applicator components against contamination and may be disposable or reusable. Also included are tissue acquisition features including a tissue receiving chamber defined by a bio-barrier with vacuum ports or channels for tissue acquisition. Vacuum balancing is provided to prevent contamination on the applicator side of the bio-barrier. Locking mechanisms are disclosed for ensuring secure attachment between the interface and applicator. Methods of using the applicator-tissue interface in connection with an applicator are also disclosed.

### RELATED APPLICATION DATA

This application claims priority to U.S. Provisional Patent Application Ser. No. 61/208,315, filed Feb. 23, 2009, and entitled “Systems, Apparatus, Methods And Procedures For The Non-Invasive Treatment Of Tissue Using Microwave Energy,” which is expressly incorporated herein by reference in its entirety.

This application also claims priority to and is a continuation-in-part of PCT Application Serial No. PCT/U.S. 2009/002403 filed Apr. 17, 2009 designating the US, and entitled “Systems, Apparatus, Methods and Procedures for the Non-Invasive Treatment of Tissue Using Microwave Energy,” which is expressly incorporated herein by reference in its entirety.

This application also claims priority to co-pending provisional U. S. Patent Application Ser. No. 61/279,153 filed Oct. 16, 2009, and entitled “Systems, Apparatus, Methods, and Procedures for the Non-Invasive Treatment of Tissue Using Microwave Energy,” which is expressly incorporated herein by reference in its entirety.

This application also claims priority to and is a continuation-in-part of PCT Application Serial No. PCT/U.S. 2009/005772 filed Oct. 22, 2009 designating the US, and entitled “Systems, Apparatus, Methods and Procedures for the Non-Invasive Treatment of Tissue Using Microwave Energy,” which is expressly incorporated herein by referenced in its entirety.

This application is further a continuation-in-part of: PCT Application Ser. No. PCT/U.S. 2008/013650, filed Dec. 12, 2008 designating the US, and entitled “Systems, Apparatus, Methods and Procedures For The Non-Invasive Treatment Of Tissue Using Microwave Energy;” U.S. patent application Ser. No. 12/450,861, filed Apr. 18, 2008, and entitled “Systems and Methods For Creating an Effect Using Microwave Energy To Specified Tissue;” U.S. patent application Ser. No. 12/450,860, filed Apr. 18, 2008, and entitled “Methods, Devices, and Systems for Non-Invasive Delivery of Microwave Therapy;” U.S. patent application Ser. No. 12/450,859, filed Apr. 18, 2008, and entitled “Methods and Apparatus for Reducing Sweat Production;” and U.S. patent application Ser. No. 12/107,025, filed Apr. 21, 2008, and entitled “Systems and Methods For Creating an Effect Using Microwave Energy To Specified Tissue.” Each of the above co-pending US and international applications designating the US are expressly incorporated herein by reference in their entirety.

### FIELD OF THE INVENTION

The present application relates to methods, apparatuses, and systems for the non-invasive delivery of treatments to tissue, including treatments based on energy delivery to tissue. In particular, the present application relates to methods, apparatuses, and systems for the interface between a treatment delivery device and the tissue, including disposable devices such as covers for devices including additional functionality to facilitate treatment.

### BACKGROUND

It is known that energy-based therapies can be applied to tissue throughout the body to achieve numerous therapeutic and/or aesthetic results. There remains a continual need to improve on the effectiveness of these energy-based therapies and provide enhanced therapeutic results with minimal adverse side effects or discomfort. One aspect for improvement of existing systems and methods includes the tissue/device interface and disposable systems for use therewith.

### SUMMARY OF THE DISCLOSURE

Systems and methods apply, in a non-invasive manner, energy to a targeted tissue region employing a controlled source of energy, an applicator, and an applicator-tissue interface carried by the applicator. The systems and methods can generate and apply energy in a controlled fashion to form a predefined pattern of lesions that provide therapeutic benefit, e.g., to moderate or interrupt function of the sweat glands in the underarm (axilla). To facilitate application of treatment to the tissue a tissue interface system may be employed, acting between the delivery device and the tissue to be treated. Such an interface system can be configured as a disposable cover for the treatment delivery device or the delivery component thereof.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a perspective view of a system for applying, in a non-invasive manner, forms of energy to body tissue to achieve desired therapeutic and/or aesthetic results comprising a console, an applicator, and an applicator-tissue interface.

FIGS. 2 and 3 are perspective views of an applicator and applicator-tissue interface shown in FIG. 1, with FIG. 2 showing a disposable applicator-tissue interface joined to the applicator for use and FIG. 5 showing the applicator-tissue interface detached from the applicator prior to or after use.

FIG. 4 is an exploded perspective view of an exemplary disposable applicator-tissue interface as shown for example in FIG. 3.

FIG. 5 is an assembled side section perspective view of the applicator-tissue interface shown in FIG. 4.

FIGS. 6A and 6B are top and bottom plane views of a disposable applicator-tissue interface such as shown in FIGS. 4 and 5.

FIG. 7 is a perspective view of a disposable applicator-tissue interface according to an alternative embodiment of the invention.

FIG. 8 is a cutaway side view of a disposable applicator-tissue interface according to an embodiment of the invention as viewed along line A-A in FIG. 7.

FIG. 9 is a cutaway side view of a disposable applicator-tissue interface according to an embodiment of the invention as viewed along line B-B in FIG. 7.

FIG. 10 is an assembled, cross-sectional interior view of an exemplary applicator, for example as shown in Figs.1-3, which may be used in connection with disposable applicator-tissue interfaces according to embodiments of the invention.

FIG. 11 is a bottom view, partially broken away, of an exemplary treatment surface, illustrating, in this embodiment a waveguide antenna array waveguide cradle, and cooling path.

FIG. 12 is a partial, cut-away perspective view of an applicator-tissue interface according to an embodiment of the invention attached to an exemplary applicator as in use.

FIG. 13 is an enlarged detailed inside, side view of the body member wall and vacuum channel inside an exemplary applicator-tissue interface as viewed at detail A in FIG. 6A.

FIG. 14 is a schematic side section view of a generic applicator with an embodiment of a disposable applicator-tissue interface according to the invention illustrating tissue acquisition.

FIG. 15 is a perspective view of a system connection including an exemplary vacuum trap for coupling a disposable applicator-tissue interface to a system console as shown in FIG. 1.

FIG. 16 is a cross-sectional view of an exemplary vacuum trap as in FIG. 15.

FIGS. 17A and 17B are views of representative treatment templates for use in methods and procedures according to the present invention.

FIG. 18 is a perspective view of packaging for an applicator and/or disposable applicator-tissue interface according to embodiments of the present invention.

### DETAILED DESCRIPTION

This Specification discloses various systems and methods for an interface between tissue to be treated and an applicator applying, in a non-invasive manner, forms of energy to body tissue to achieve desired therapeutic and/or aesthetic results. Such an applicator-tissue interface may be conveniently provided a disposable component, however disposability is not required. As described, the systems and methods are particularly well suited for treating the epidermal, dermal, and sub-dermal tissue of an individual to treat, e.g., skin conditions, aesthetic conditions, glandular structures, vascular structures, hair follicles or other conditions. For this reason, the systems and methods will be described in this context, and, in particular, in the context of the application of electromagnetic microwave energy to sweat glands to treat hyperhidrosis, or excessive sweating. However, with suitable modifications as will be apparent to persons of ordinary skill in the art, applicator-tissue interface systems according to embodiments of the invention as described herein may be used with different treatment modalities.

Further, although the disclosure contained in this Specification is complete with respect to the described exemplary embodiments to enable those skilled in the art to practice the invention, the physical embodiments disclosed are intended to exemplify representative embodiments that highlight the technical features of the invention. The technical features of the invention may be embodied in other specific structures. While certain exemplary embodiments have been described, the details may be changed without departing from the technical features of the invention as defined in the claims.

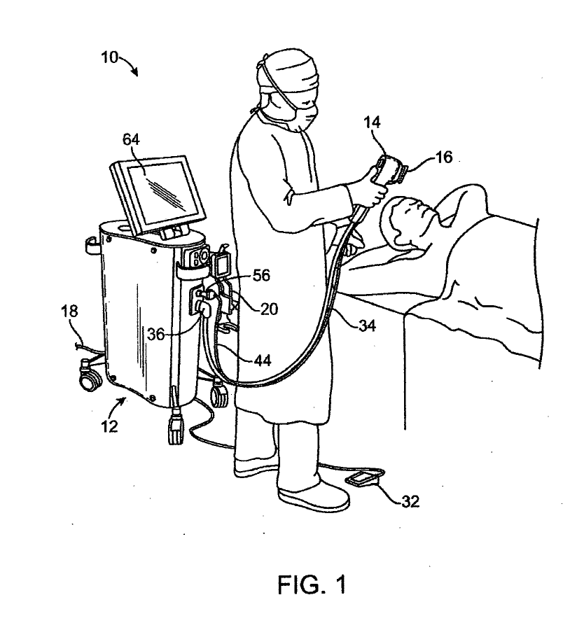

FIG. 1 shows an exemplary system 10 for applying, in a non-invasive manner, energy to a targeted tissue region that embodies the features of the invention. As shown in FIG. 1, the exemplary system 10 includes three general components. These are a system console 12, a system applicator 14, and an applicator-tissue interface 16 carried by the system applicator 14. If desired, applicator-tissue interface 16 may be a disposable component.

In the illustrative embodiment shown in FIG. 1, system 10 is particularly sized and configured to generate and apply energy to the underarm (axilla) of an individual to form a predefined pattern of lesions. The pattern of lesions serves, e.g., to moderate or interrupt function of the sweat glands in the underarm. In this illustrative arrangement, the system 10 and its method of use can serve to treat, e.g., axillary hyperhidrosis or underarm sweating/odor. While an applicator using microwave energy to form lesions for treatment is used as an example to illustrate embodiments of the invention, other applicators may be employed with embodiments of the invention. Further details of microwave specific embodiments may be found in the above cited priority applications, including, but not limited to U.S. Provisional Patent Application Ser. No. 61/208,315, filed Feb. 23, 2009, and entitled “Systems, Apparatus, Methods And Procedures For The Noninvasive Treatment Of Tissue Using Microwave Energy,” and PCT Application No. PCT/U.S. 2009/002403 filed Apr. 17, 2009 and designating the US, entitled “Systems, Apparatus, Methods and Procedures for the Noninvasive Treatment of Tissue Using Microwave Energy,” each of which is expressly incorporated herein by reference in its entirety and particularly with respect to that disclosure.

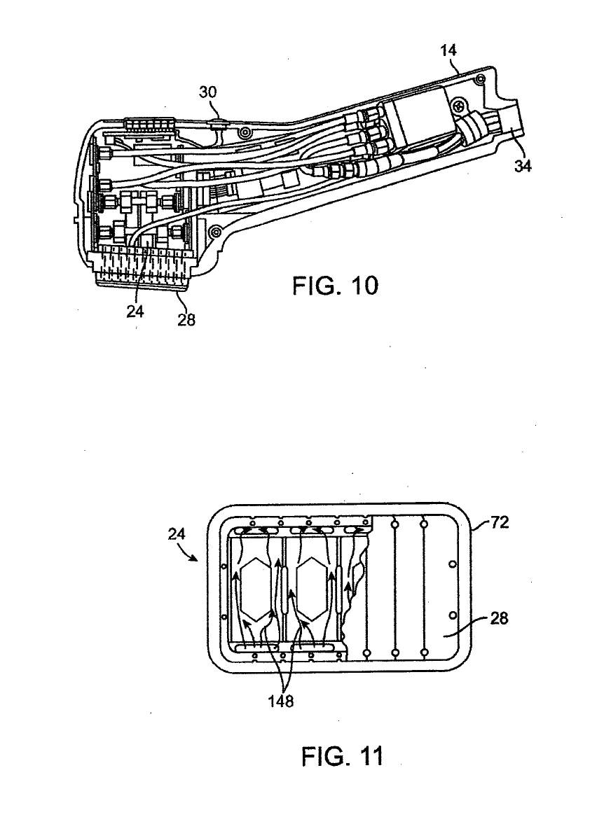

A typical system applicator 14 may be a durable item capable of repeated re-use. Such a system applicator 14 may be sized and configured to be, during use, conveniently handled and manipulated in a hand of a caregiver. As shown in FIGS. 1-3, system applicator 14 may comprise a pistol-grip housing made, e.g., of molded plastic material. Other shapes are possible. Treatment delivery components may be carried within the housing, such as a waveguide antenna array 24 as in the illustrated embodiment (see, e.g. FIG. 10).

Components in the applicator 14 also act in concert with components housed within the system console 12 to carry out a desired treatment such as energy delivery for lesion generation and control functions. A “trigger” switch 30 on the system applicator 14, which may, for example, be thumb actuated, can give the caregiver direct control over initiation and termination of treatment, subject to the overrides and global control of the master controller of the system console 12. Alternatively, or in combination, a foot pedal control switch 32 can be provided for the same purpose (see FIG. 1). A special purpose cable assembly 34 couples the components housed in the system applicator 14 to the components housed within the system console 12. The special purpose cable assembly 34 includes a custom designed multi-function plug 36 that couples to a dedicated connection 38 site on the system console 12.

In general, an applicator for use with embodiments of the present invention, such as applicator 14 will include a treatment surface through which the desired treatment modality, e.g. microwave energy, is delivered to the tissue. An exemplary treatment surface 28 is shown for applicator 14 in FIGS. 10 and 11. In the illustrated embodiments treatment surface 28 comprises a flat plate structure configured to be placed against or in close proximity to the tissue to be treated. Other shapes or configuration of treatment surface 28 are possible without departing from the scope of the invention. As is known in the art, it is often desirable to protect such a treatment surface against contamination by the tissue being treated. This may be particularly necessary where the treatment surface is part of a more complex applicator that is not readily sterilized. In such instances it is known to provide a cover to protect the treatment surface, which cover may be sterilizable itself or disposable.

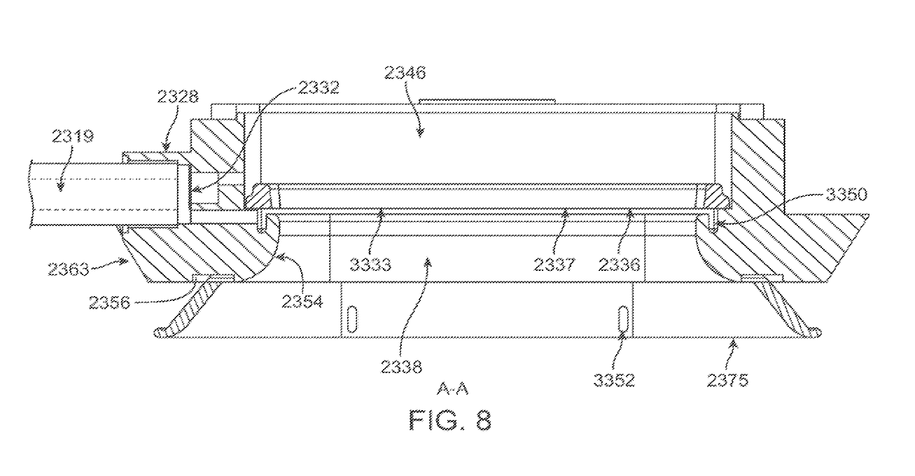

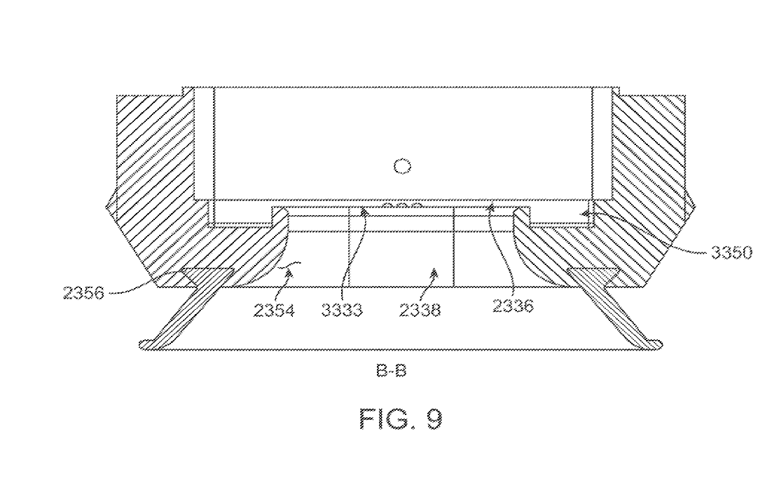

Such a cover according to embodiments of the present invention comprises applicator-tissue interface 16. The applicator-tissue interface 16 may be a single use, disposable item. Specific materials described herein below are suitable for a disposable item. However, persons of ordinary skill in the art may select appropriate sterilizable and re-useable materials without departing from the scope of the invention. More particularly, as shown in FIGS. 2-9, interface 16 may be sized and configured to be temporarily coupled to the system applicator 14 during use (e.g., by a latching mechanism 40), and then detached after use for disposal. In this arrangement, the applicator-tissue interface 16 can, after an incidence of use, be detached from the system applicator 14, discarded, and replaced by another unused applicator-tissue interface 16 prior to a next incidence of use.

In use, the applicator-tissue interface 16 contacts the targeted tissue region and passes the energy radiated by the applicator, and particularly the treatment surface 28, to tissue. Components in the applicator-tissue interface 16 also act in concert with components housed within the system console 12 to carry out the tissue acquisition function. For this purpose, the applicator-tissue interface 16 includes a tissue acquisition chamber 42, into which tissue is drawn to elevate the dermis and hypodermis and localize and stabilize the targeted tissue region. It may also be desirable to bring the tissue into thermal conductive contact with treatment surface 28 as energy is applied from applicator 14. In the illustrated embodiment, the tissue acquisition function includes the application of a vacuum to the tissue acquisition chamber acquisition chamber 42. For this purpose, a vacuum supply conduit 44 couples the components housed in the applicator-tissue interface 16 to components housed within the system console 12. The vacuum supply conduit 44 plugs into a dedicated connection site 48 on the system console 12 or in a general service vacuum part.

The application of the vacuum by the applicator-tissue interface 16, as controlled by the tissue acquisition function, provides uniformity and consistency in acquiring tissue for treatment. It reduces variability of treatment that may arise, e.g., due to differences in manipulation of the applicator by a given caregiver and/or difference among tissue topologies to be treated. Details of a specific vacuum system may be found in the above incorporated applications.

The applicator-tissue interface 16 also includes a multi-functional bio-barrier including at least three bio-barrier components: a first membrane 52 that is liquid and gas impermeable, a second membrane 54 that is liquid impermeable but gas permeable and a vacuum trap 56. As will be described in greater detail later, the multi-functional bio-barrier isolates the operational components in the applicator 14 and the console 12 from contact with and contamination by physiologic liquids (e.g., blood and sweat) that may be present in the targeted tissue region. The multi-functional bio-barrier substantially isolates the durable electrical and mechanical components of the system 10 (e.g., the applicator 14 and console 12), from the physiologic conditions of the tissue regions being treated, and vice versa.

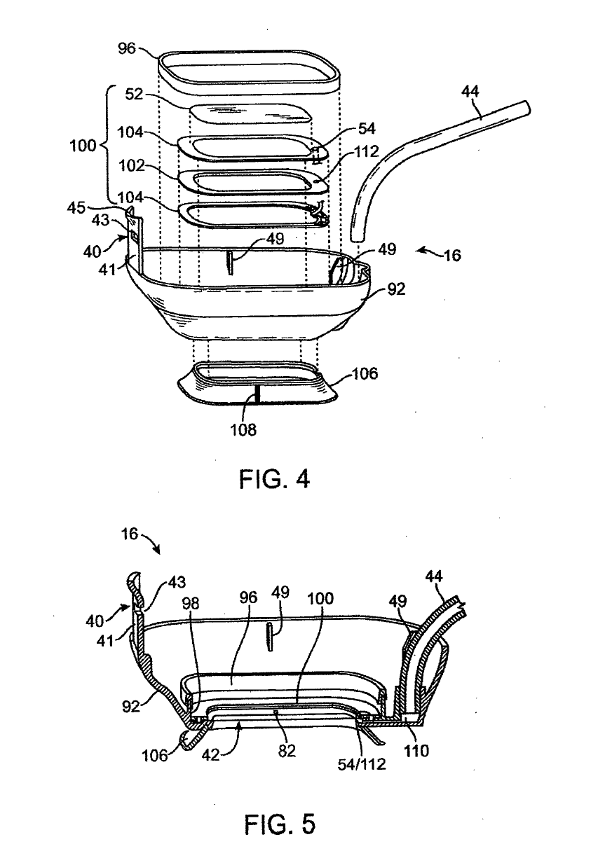

Referring now to FIGS. 4 and 5, the applicator-tissue interface 16 may comprise a body 92 formed from a medical grade rigid or semi-rigid plastic material, e.g., polycarbonate. The body 92 may be formed, e.g., by molding, into a bowl shape. Latching assembly 40 can be integrally formed on the body 92 to couple to a mating attachment member 94 on the system applicator 14, to fasten the applicator-tissue interface 16 to the system applicator 14 at time of use and disconnect the interface from the system applicator 14 after use.

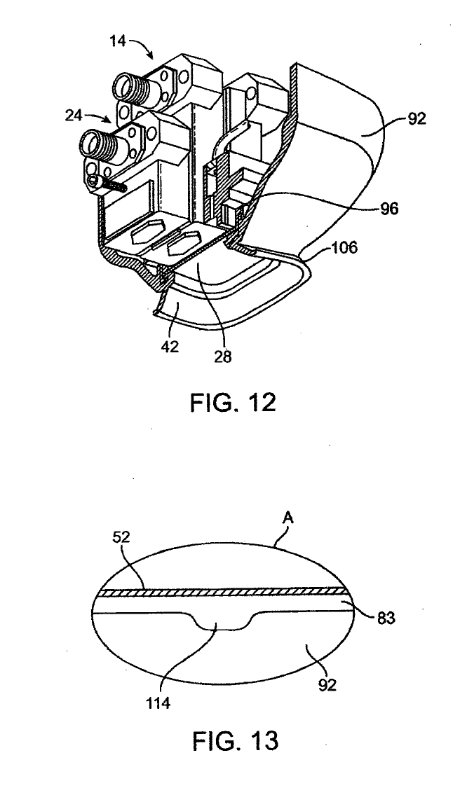

Within the bowl shaped body 92, an applicator gasket 96 is seated on peripheral flange 98 formed in the bowl. The applicator gasket 96 is sized and configured, when the interface body 92 is fastened to the system applicator 14 (see e.g. FIG. 12), to form a fluid-tight, pressure-tight seal against the periphery of the applicator around or immediately behind treatment surface 28 on the undersurface of applicator 14.

Within the bowl shaped body 92, spaced below and inward of the applicator gasket 96, is a tissue interface surface 100. In the illustrated exemplary embodiment, the tissue interface surface 100 comprises a frame 102 with upper and lower overlying adhesive panels 104. A first bio-barrier component or membrane 52 is mounted on the upper adhesive panel, and the lower adhesive panel adheres to an interface surface support in the bowl, which the applicator gasket 96 peripherally surrounds.

In use, tissue being treated contacts the first bio-barrier component 52 in thermal contact with at least a portion of the treatment surface 28. For this reason it may be desirable that first bio-barrier component 52 at least substantially closely adheres to the treatment surface 28 of substantially the entire area of the treatment surface. The first bio-barrier component 52 forms a part of the multi-functional bio-barrier of the applicator-tissue interface 16. The first bio-barrier component 52 comprises the actual tissue surface interface, which tissue, when acquired within the tissue acquisition chamber 42, contacts as energy or other treatment is applied from the treatment surface 28. Component 52 also acts as a treatment window through which treatment is delivered. The first bio-barrier comprises 52 a material that is selected on the basis of different, but overlapping physical criteria.

One selection criterion for the first bio-barrier component 52 is that the material is substantially impermeable to both air and liquids, such as blood and/or sweat, which may be present in the tissue acquisition chamber 42. As the tissue acquisition function applies vacuum to draw tissue within the tissue acquisition chamber 42 into contact with the first bio-barrier component 52, the first bio-barrier component 52 isolates the components in the applicator 14 from contact with and contamination by physiologic liquid in the targeted tissue region.

An overlapping selection criterion for the first bio-barrier component 52 is that the material, taking into account its thickness, possesses characteristics necessary to adequately pass the energy used as the treatment modality. For example, where microwave energy is the treatment modality a low microwave conductivity is required, so that component 52 efficiently passes the microwave energy radiated by the waveguide antenna array 24 to the targeted tissue region acquired within the, tissue acquisition chamber 42, with minimal energy absorption. For microwave energy deliver, the characteristic can be expressed as a loss tangent tan σ of 0.1 or less, and more desirably approximately 0.0004.

The loss tangent tan δ is similar to conductivity a, but also takes into account the dielectric constant of the material, as follows:

tan δ=σ/ωε

where ω is frequency, and where ε is permittivity

For example, at 5.8 GHz, a range of conductivities a suitable for use as the first bio-barrier component 52, corresponding to a tan δ equal to or less than 0.1, would be σ=0.0 to 0.2 siemens/meter.

Another overlapping selection criterion for the first bio-barrier component 52 may be the thermal conductivity when thermal energy is a part of the treatment modality. In such an instance, it may be necessary that the material, taking into account its thickness, possess a requisite high thermal conductivity, to efficiently allow thermal conduction to occur between the targeted tissue region acquiring within the tissue acquisition chamber 42 and the treatment surface 28. Again, considering the example of a microwave treatment modality, the material selected should have a thermal conductivity of at least 0.1 watts per meter=Kelvin (0.1 W/mK), and desirably 0.1 to 0.6 W/mK, and most desirably 0.25 to 0.45 W/mK.

Another overlapping selection criterion for the first bio-barrier component 52 that may be necessary to consider when thermal energy delivery is a part of the treatment modality is that the material, taking into account its thickness, possesses requisite high heat transfer coefficient. The heat transfer coefficient can be expressed by the thermal conductivity of the material divided by the thickness of the material. For example, for a first bio-barrier component 52 with a thermal conductivity of 0.1 and a thickness of 0.0005 inches, the heat transfer coefficient would be about 7874 W/m2K.

Other overlapping selection criteria for the first bio-barrier component 52 include that the material is sufficiently flexible to conform to the treatment surface 28, while also being sufficiently strong to resist tearing as a result of vacuum pressure or contact with tissue. In one exemplary embodiment, the membrane material should be of sufficient strength to withstand a vacuum pressure of at least about −20 mm Mercury +/−1. An example of flexibility is that it should be able to sufficiently adhere to the treatment surface under such a vacuum pressure so as to eliminate any bubbles between the membrane and treatment surface that would exceed about 0.003 inches in diameter. In general, when conduction of energy directly through bio-barrier component 52 is a part of the treatment modality it is desirable that uniform adherence across the treatment surface 28 be achieved without the presence of trapped bubbles. Another material characteristic that may be desirable is the deformability of the membrane material used as bio-barrier component 52 so that adherence can be achieved even when the treatment surface is not necessarily initially contacting the membrane before vacuum application. In this regard, the ability to deform a distance of at least about 1 mm over substantially the area of the treatment surface may be desirable.

With respect to materials selection, the first bio-barrier component 52 may be a nonporous membrane, e. g., polyethylene film, nylon, Mylar, or other suitable materials. The first bio-barrier component 52 is also desirably flexible and soft for compliant contact with skin. Considering these characteristics, the first bio-barrier component 52 may comprise, e.g., polyethylene film available from Fisher Scientific, or (alternatively) Mylar film. The polyethylene film in particular is well suited for microwave applications. In such an exemplary embodiment, the bio-barrier component 52 may be, e.g., about 0.0005 inch in thickness.

The applicator-tissue interface body 92 also includes a skirt 106 that depends downwardly with an increasing diameter from the body about the periphery of the applicator-tissue interface surface. The downward depending skirt 106 defines a generally funnel-shaped open interior area or chamber leading to the first bio-barrier component 52 of the applicator-tissue interface 16. This chamber defines the tissue acquisition chamber 42 previously described. The skirt 106 may comprises compliant medical grade plastic material (e.g., a thermal plastic elastomer (TPE) such as urethane; or silicone; or natural or synthetic rubber; or an elastomeric material) and may be sized and configure to rest comfortably against an external skin surface. When pressed with sufficient pressure to compress against a tissue surface (see e.g. FIG. 14), the periphery of the skirt 106 forms a generally fluid-tight, pressure-tight seal about the tissue acquisition chamber 42.

The skirt 106 may include an alignment marker 108 positioned on each side of the skirt 106 to provide a positioning-point of reference to the caregiver during manipulation of the interface, as will be described in greater detail later. Such an alignment marker may be usefully positioned in approximately the center of the skirt 106 along the long dimension side. Other locations may be well suited for other treatment modalities. The funnel-shaped contour of the skirt 106 may provide a skirt angle that gives the caregiver a direct view of the alignment members 108, while the caregiver manipulates the applicator-tissue interface 16 attached to the system applicator 14.

In one exemplary embodiment, alignment marker is formed by first notching the skirt 106 and then filling the notch with an opaque adhesive material. Other forms of printing or embossing may be used.

Vacuum supply conduit 44, communicating with the tissue acquisition function of the system console 12, is coupled to a port formed on the body of the applicator-tissue interface 16. The port communicates with a vacuum channel 110 formed in the body that communicates with the tissue acquisition chamber 42 adjacent the applicator-tissue interface surface. The vacuum channel 110 may circumferentially encircle the tissue acquisition channel at or near the applicator-tissue interface surface. The vacuum channel 110 may include spaced-apart apertures or ports 82 formed along the vacuum channel (e.g. four ports, one adjacent each side the applicator-tissue interface surface), to convey negative pressure uniformly into the tissue acquisition chamber 42 adjacent the applicator-tissue interface surface. The ports 82 suction skin into the chamber and position the skin against the first bio-barrier component 52 in thermal conductive contact with the treatment surface 28 (as FIG. 21B shows).

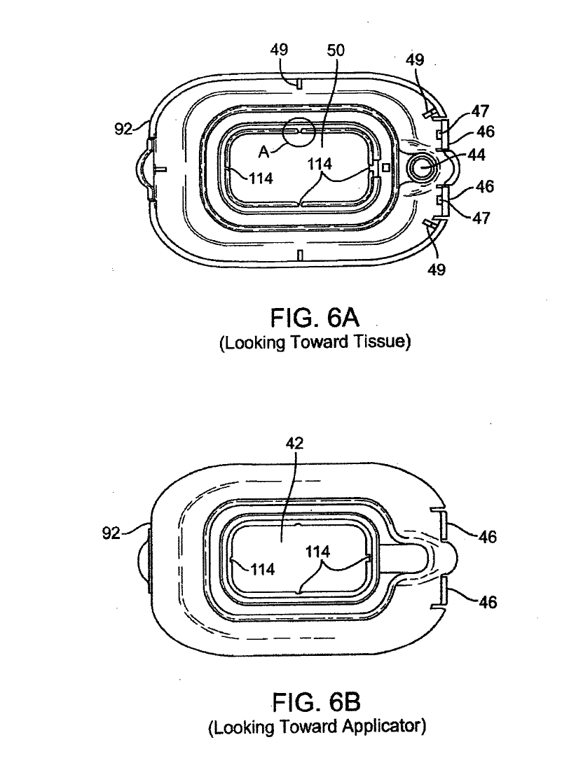

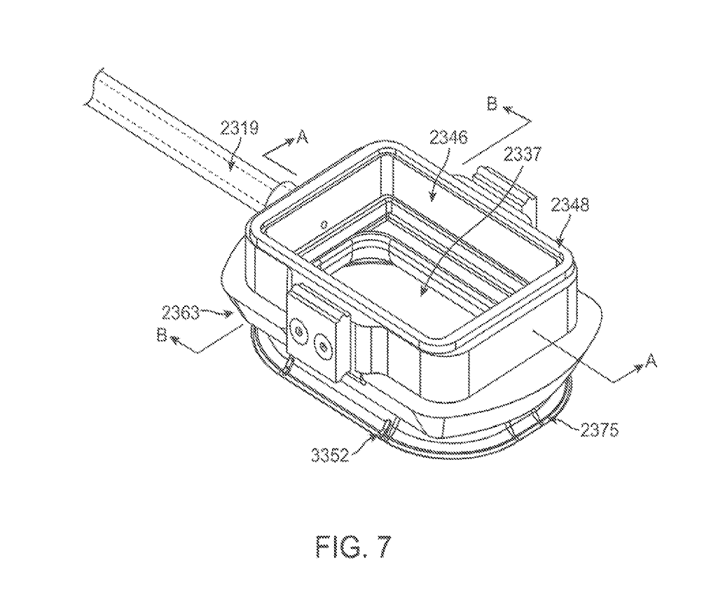

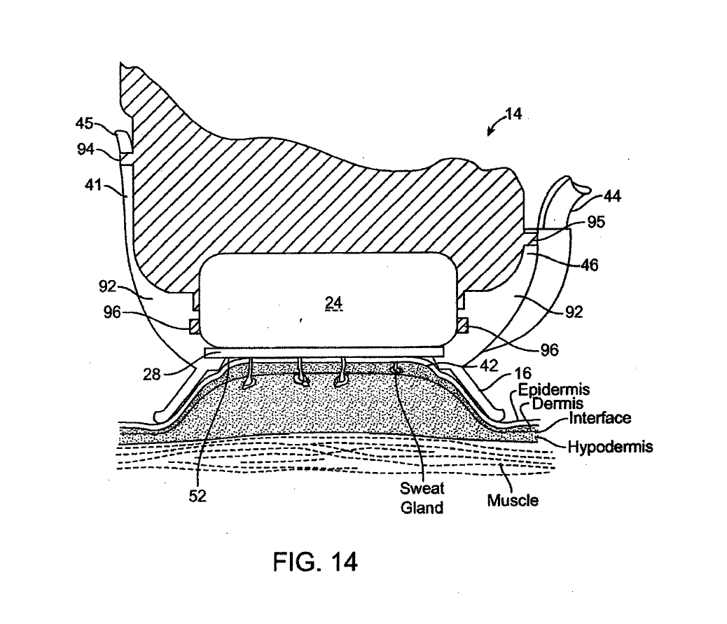

As an alternative to ports 82, a narrow, slit-like opening may extend around the vacuum channel to form a continuous “port” as shown in more detail in the embodiment of FIGS. 7-9. As a further alternative shown in FIGS. 6A, 6B and 13, a continuous, slit-like port 83 is provided with notches 114 for increased suction and to help avoid blockages from loose tissue or foreign matter. Notches 114 may be distributed evenly around membrane 52.

The frame 102 and panels 104 of the tissue interface surface 100/52 may include formed apertures 112 (see e.g. FIGS. 4 and 5) that register when assembled to form a vacuum balance path that communicates with the tissue acquisition chamber 42. Negative pressure applied in the chamber 42 is conveyed through the vacuum balance path 112 to the opposite side of the interface surface 100/52 to equalize pressure on both sides of the interface surface 100/52.

A second bio-barrier component 54 of the multi-functional bio-barrier 50 of the applicator-tissue interface 16 desirably occupies the vacuum balance path 112. The second bio-barrier component 54 in the vacuum balance path 112 (which can also be called the “vacuum balance bio-barrier component” comprises a material that is substantially impervious to liquid, but not to air. The vacuum balance bio-barrier component 54 prevents physiologic liquids such as blood and/or sweat that may be present in the tissue acquisition chamber 42 from being transported through the vacuum balance path 112 into the interior of the applicator 14. Candidate materials for the second bio-barrier component 54 may include pores sufficient to pass air (e.g., 0.45 um) to substantially equalize the vacuum pressure on the system applicator side and the interface side of the surface, without passing biological liquids from the acquisition chamber 42 into the system applicator 14. The second bio-barrier component 54 may comprise, e.g., a hydrophobic membrane made from PTFE (Teflon) material. The second bio-barrier component 54 can be, e.g., about 0.005 inch in thickness.

In an exemplary embodiment particularly suited for microwave application, the tissue acquisition chamber 42 is dimensioned approximately 1.54 inches by approximately 0.7 inches, having a depth (without the skirt 106) of approximately 0.177 inch (4.5 mm). With the skirt 106, the depth of tissue acquisition chamber 42 can be between approximately 6.5 mm to 11 mm, depending upon the extent to which the compliant skirt 106 is compressed against the skin by the application of vacuum. According to an embodiment of the invention the four corners of the tissue acquisition chamber 42 may have a radius of about 0.1875 inches. The tissue acquisition chamber 42 is desirably optimized to facilitate tissue acquisition without adversely impacting cooling or energy transmission.

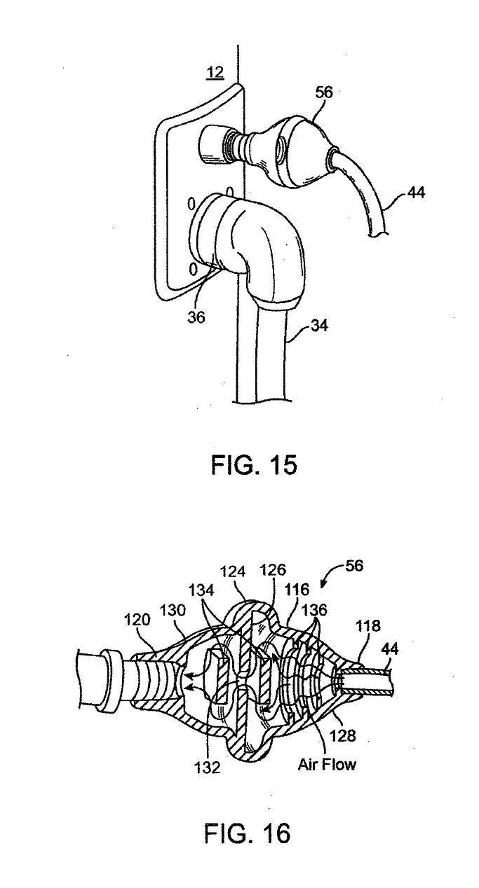

The vacuum supply conduit 44 may collect liquids (e.g., sweat or blood) that escape during the treatment process. For this reason, a third bio-barrier component 56 of the multi-functional bio-barrier 50 of the applicator-tissue interface 16 is placed upstream of the applicator-tissue interface 16 in-line in the vacuum supply conduit 44 (see. FIGS. 15 and 16 also generally shown in FIG. 1). The third bio-barrier component 56 is selected to be substantially impervious to liquid, but not to air. The third bio-barrier component 56 can comprise, e.g., a hydrophobic filter (e.g., a Millex FH filter made of 0.45 11 m hydrophobic PTFE available from Millipore) to keep liquids out of the system console 12. The hydrophobic filter can be further characterized, e.g., by accommodating an airflow of approximately 13.4 cubic feet per minute at approximately 10 pounds per square inch.

The third bio-barrier component 56 can, alternatively, comprise an in-line vacuum trap, as shown in FIGS. 16C and 16D. The vacuum trap may include a formed housing 116 defining a vacuum inlet port 118 (with which the vacuum supply conduit 44 communicates) and a vacuum outlet port 120 (which plugs into the connection site 48 on the console 12). The housing 116 defines an interior chamber 122, which the vacuum flow between the inlet and outlet ports 118 and 120 must traverse from the applicator-tissue interface 16 to the system console 12. A central ridge 124 on the exterior of the housing 116 may provide a gripping surface for the caregiver to hold and manipulate the vacuum trap, e.g., while plugging the vacuum supply connector 118 into and out of the mating console vacuum supply receptacle 48.

The chamber 122 is compartmentalized by an interior wall 126 into an inlet side 128, communicating with the inlet port 118, and an outlet side 130, communicating with the outlet port 120. One or more apertures 132 in the interior wall 130 define path(s) of flow communication between the inlet and outlet sides 128 and 130 of the chamber 122.

Baffle plates 134 interfere with vacuum flow through the aperture(s) 132 through the interior wall 16 between the inlet side 128 and outlet side 130 of the chamber 122. The vacuum flow must veer around the baffle plates 134 to transit through the chamber 122. An array of annular baffles 136 is further circumferentially placed around the inlet side 128 of the chamber 122. The baffle plates 134 and annular baffles 136 form an array of tortuous paths, through which vacuum flow transiting the chamber must navigate. Air in the vacuum flow will readily change direction to navigate the tortuous paths. Physiologic liquid carried by the vacuum flow will not, and will instead be captured by gravity in the nooks and crannies of the tortuous paths through the chamber 122. The vacuum trap thereby prevents physiologic liquid from passing out of the outlet port 120 into the console 12.

Locking mechanism 40 for securing applicator-tissue interface 16 to an applicator will now be described in more detail in connection with FIGS. 2-4, 6A and 14. In an exemplary embodiment, mechanism 40 includes upwardly extending member 41 at the front of the applicator-tissue interface 16 with opening 43 configured to receive a corresponding element 94 on applicator 14. Member 41 may be resilient so as to snap over element 94 and may include ramped portion 45 for ease of attachment. At the rear of body member 92, two tab members 46 are provided (see, e.g. FIG. 6A). Tab members 46 may be formed in the body member wall, terminating at the top edge, delimited by slots on either side and joined to the wall at the bottom. Tab members 46 each have openings 47 configured to receive corresponding elements 95 formed on the back of the applicator 14. Further facilitating proper connection between the applicator 14 and applicator-tissue interface 16 are guide or alignment ribs 49.

A further alternative embodiment of an applicator-tissue interface according to the invention is shown in FIGS. 6-8. In this exemplary embodiment of the invention, applicator-tissue interface 2363 may include tissue interface surface 2336, tissue chamber 2338 and alignment features 3352. Tissue interface surface 2336 may form a back wall of tissue chamber 2338. Tissue interface surface 2336 may include first bio-barrier 2337 and vacuum channel 3333. Vacuum channel 3333 may also be referred to as a lip or rim. According to an embodiment of the invention applicator-tissue interface 2363 may include alignment features 3352 and vacuum tubing 2319. According to an embodiment of the invention applicator-tissue interface 2363 may include compliant member 2375. Compliant member 2375 may be formed from a compliant material, such as, for example, rubber, coated urethane foam (with a compliant plastic or rubber seal coating), silicone, polyurethane or heat sealed open cell foam. According to one embodiment of the invention, compliant member 2375 may be positioned around the outer edge of tissue chamber 2338 to facilitate the acquisition of tissue. Compliant member 2375 also may facilitate the engagement of tissue which is not flat, such as, for example tissue in the axilla, and thus may speed the acquisition of tissue in tissue chamber 2338.

According to an exemplary embodiment of the invention, compliant member 2375 may have a height of between approximately 0.15 inches and approximately 0.40 inches and. more specifically approximately 0.25 inches above chamber 2338 opening when compliant member 2375 is not compressed. Alignment features 3352 may be positioned at a distance which facilitates appropriate placement of applicator 2320 during treatment; for example, they may be positioned approximately 25-35 millimeters apart. In a further embodiment, an outer edge of compliant member 2375 may assist a user in aligning medical treatment device 2300.

According to a further embodiment of the invention compliant member 2375, which may also be referred to as a skirt or flexible skirt, may be manufactured from a resilient material such as silicone and may have a durometer density rating (softness) of approximately A60 which may help compliant member 2375 to maintain its shape better while being easier to mold. A counter sink or dovetail notch 2356 may be formed in the rigid body member around the outer edge of chamber opening 2339 to assist in alignment of compliant member 2375. If desired, a colorant may be used in compliant member 2375 to contrast with skin viewed through compliant member 2375, making it easier for user, such as a physician to distinguish between skin and a distal surface of compliant member 2375. The angle of compliant member 2375 relative to the surface of the body member where attached may be approximately 53 degrees when compliant member 2375 is not compressed.

Disposable applicator-tissue interface 2363 also includes an applicator chamber 2346 which may be formed, at least in part, by tissue bio-barrier 2337. Bio-barrier 2337 may be, for example, a polyethylene film, available from Fisher Scientific or it may be a Mylar film. According to an embodiment of the invention a counter bore may positioned between applicator bio-barrier 2332 and applicator chamber 2346.

According to an embodiment of the invention vacuum passage 3333 connects vacuum channel 3350 to tissue chamber 2338. Vacuum channel 3350 may also be referred to as a reservoir or vacuum reservoir. Vacuum connector 2328 is connected to vacuum passage 3333 through vacuum channel 3350. Vacuum passages 3333 form a direct path to tissue interface surface 2336. Vacuum passages 3333 and vacuum channel 3350 may be adapted to restrict the movement of fluids from tissue chamber 2338 to applicator bio-barrier 2332. Vacuum connector 2328 may be positioned on the same side of applicator-tissue interface 2363 as applicator bio-barrier 2332. Applicator bio-barrier 2332 may be designed to prevent fluids from tissue chamber 2338 from reaching applicator chamber 2346, particularly when there is back pressure caused by, for example, a vacuum created in tissue chamber 2338 as tissue is pulled away from tissue interface surface 2336. Vacuum channel 2350 may surround tissue interface surface 2336. Applicator bio-barrier 2332 may be positioned between vacuum passages 3333 and applicator chamber 2346.

Applicator bio-barrier 2332 may be a membrane which may be adapted to be permeable to air but substantially impermeable to biological fluids such as, for example, blood and sweat. According to an embodiment of the invention applicator bio-barrier 2332 may be a hydrophobic membrane filter. According to a further embodiment of the invention, applicator bio-barrier 2332 may be made of polyethylene film, nylon or other suitable materials. Applicator bio-barrier 2332 may include pores having sizes sufficient to pass enough air to substantially equalize the vacuum pressure in applicator chamber 2346 and in tissue chamber 2338 without passing biological fluids from tissue chamber 2338 to applicator chamber 2346. For example, applicator bio-barrier 2332 may include pores having sizes of approximately 0.45 micrometers.

When the vacuum is turned on, and before pressure is equalized, applicator bio-barrier 2332 may induce a minimal pressure drop between vacuum passages 3333 and the applicator chamber 2346. Applicator chamber 2346 and tissue chamber 2338 may be separated, at least in part, by tissue bio-barrier 2337 and tissue chamber 2338 may include tissue interface surface 2336 and chamber wall 2354.

According to an embodiment of the invention tissue chamber opening 2339 has dimensions which facilitate the acquisition of tissue. As such, tissue chamber 2339 may be sized to facilitate tissue acquisition while being large enough to prevent interference with energy radiated from the applicator through the treatment window. A vacuum circuit 3341 may include vacuum passages 3333, vacuum channel 3350 and may encircle tissue chamber 3338. According to an embodiment of the invention vacuum channel 3350 may be positioned around tissue chamber 2338. Vacuum passage 3333 may be positioned around a proximal end of tissue chamber 2338, and may be positioned between tissue bio-barrier 2337 and a proximal end of chamber wall 2354. Chamber wall may be radiused to facilitate tissue acquisition and release. In this embodiment, an opening to vacuum passage 3333 may be approximately 0.020 inches in height. According to another embodiment, an opening to vacuum passage 3333 may be approximately 0.010 inches in height when applicator tissue interface 2363 is attached to applicator 2320 and tissue bio-barrier 2337 is stretched into tissue chamber 2338 by a distal end of applicator 2320. More specifically, vacuum passage 3333 may have an opening height which is too small for tissue to invade when a vacuum is applied.

In use, to acquire tissue within applicator-tissue interface 16 or 2346, components carried in the system console 12 or other appropriate vacuum supply generate negative pressure that is communicated to the applicator-tissue interface 16 by the vacuum supply conduit 44, 2319. As described above, the applicator-tissue interface 16, 2346 includes a formed tissue acquisition chamber 42, 2338 with ports, channel and or notches through which negative pressure is directed by the vacuum supply conduit to draw tissue into the acquisition chamber. FIG. 14 illustrates tissue acquired in this manner and how the skirt member helps define the tissue acquisition chamber. The negative pressure applied to tissue in the acquisition chamber localizes and stabilizes the tissue while treatment is applied as shown. FIG. 14 illustrates a generic applicator 14 schematically in partial cross section with a treatment delivery element 24 acting through a treatment surface 28. Treatment surface 28 may comprise a plate member with cooling or other control features.

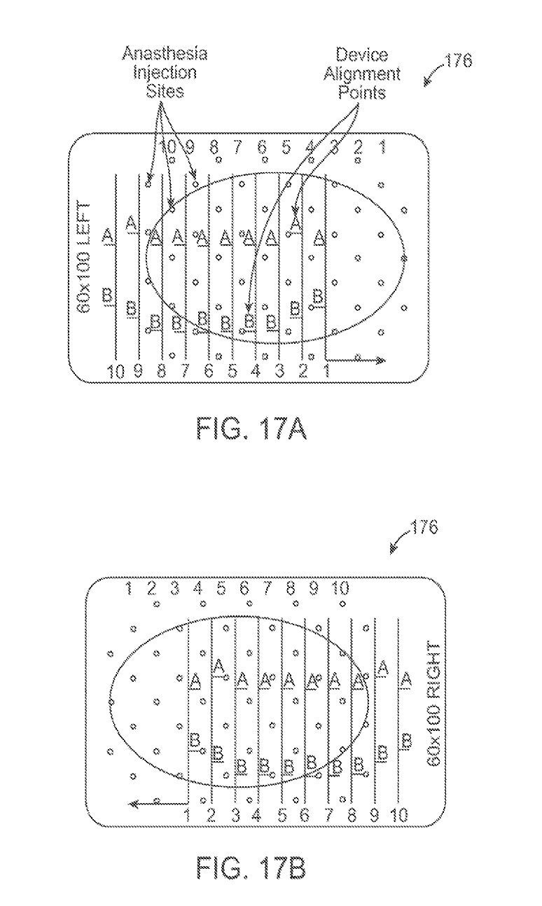

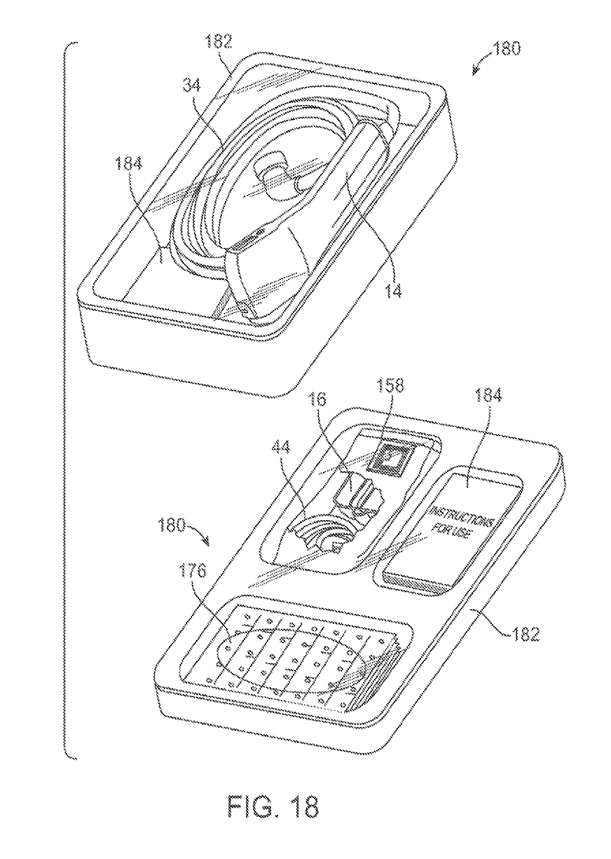

The system 10 may further include a treatment template 176 (see FIGS. 17A and 17B) to provide guidance and placement information for system applicator 14 in a matrix format. The treatment template 176 is sized and configured to overlay an area to be treated, for example the entire axilla (underarm) tissue region. The template 176 can comprise a temporary tattoo applied to each underarm. Alternatively, the template 176 can comprise a pattern applied by stamping on a tissue region. The template 176 can comprise an overlay stencil placed on the skin surface and applied by a marker pen through the stencil. The template 176 can comprise an overlay mesh sticker applied to the tissue region. A family of templates 176 (see FIG. 18) can be provided, with different sizes and arrays, to accommodate the different anatomies of individuals.

The template 176 may include prescribed anesthesia injection sites (small holes) to identify appropriate points in the axilla for the injection of anesthesia; and device alignment points in an x-y matrix axis (1A to 10A and 1B to 10B and more depending upon the size of the treatment area) to be used in conjunction with alignment markers 108 on the compliant skirt 106 to provide a positioning point of reference to the caregiver during use of the template 176.

As FIG. 18 shows, the system applicator 14 and/or applicator-tissue interface 16 of the system 10 can be provided for use in sterile kits 180. In the illustrated embodiment, each kit 180 includes an interior tray 182 made, e.g., from die cut cardboard, plastic sheet, or thermo-formed plastic material. The system applicator 14 and, applicator-tissue interface 16 is carried, by a respective tray 182. Either kit 180 can also include in the tray or separately packaged a treatment template or family of templates 176.

Each tray 182 may include a tear-away over wrap, to peripherally seal the tray from contact with the outside environment. Each kit 182 carrying the system applicator 14 and/or applicator-tissue interface 16 may be sterilized by convention ethylene oxide (ETO) sterilization techniques. In the illustrated embodiment, the packaging for one or both the system applicator 14 and/or applicator-tissue interface 16 can carry passive RFID tags 158 that interact with radio-frequency identification (RFID) source located within the applicator control system, for example on the console 12. The RFID tag may be used to ensure that a that the applicator-tissue interface 16 is properly used with the applicator 14 or that disposable applicator-tissue interfaces are not reused.

In a further exemplary embodiment, an applicator-tissue interface for use with a medical treatment device according to the present invention comprises a body member, a liquid and gas impermeable membrane, a vacuum channel, a vacuum equalization passage and a liquid impermeable gas permeable membrane. The body member has a wall surrounding a treatment opening and defining a tissue receiving chamber at a lower side and a device receiving chamber at an upper side. The liquid and gas impermeable membrane is sealingly disposed across the treatment opening between the tissue receiving chamber and the device receiving chamber to provide a bio-barrier membrane there across. The bio barrier is transparent to the treatment modality. The vacuum channel is disposed in the tissue receiving chamber adjacent the bio-barrier and surrounding the treatment opening. The vacuum channel communicates with the tissue receiving chamber to provide a negative pressure therein. The vacuum equalization passage communicates between the vacuum channel and the device receiving chamber. The liquid impermeable, gas permeable membrane is sealingly disposed across the vacuum equalization passage to prevent flow bodily fluids there through.

In other exemplary embodiments, the vacuum channel defines at least one port adjacent the treatment opening communicating with the tissue receiving chamber. The vacuum channel defines plural ports distributed around the treatment opening. The vacuum channel defines a continuous slit opening adjacent the bio-barrier surrounding the treatment opening, the slit opening communicating with the tissue receiving chamber.

According to other embodiments, the continuous slit defines at least one enlarged notch opening or the continuous slit defines plural notch openings distributed around the treatment opening. The vacuum equalization passage may comprise a port communicating directly from the vacuum channel into the device receiving chamber.

In another embodiment of the applicator-tissue interface according to the invention, the body member defines an inwardly directed flange surrounding the treatment opening; the liquid and gas impermeable bio-barrier membrane is sealing secured to the flange; and the vacuum equalization passage comprises a hole through the flange with the liquid impermeable, gas permeable membrane sealed there across.

In yet another embodiment, the body member defines a vacuum inlet communicating with the vacuum channel; the vacuum equalization passage comprises a passage communicating between the vacuum inlet and the device receiving chamber; and the liquid impermeable, gas permeable membrane is disposed in the passage.

According to a further embodiment of the applicator-tissue interface of the invention, a gasket member is disposed in the device receiving chamber around the treatment opening, the gasket member being configured and dimensioned to sealingly receive the medical treatment device with a treatment surface thereof facing the bio-barrier membrane to facilitate formation of a vacuum between the device and the bio-barrier membrane when negative pressure is applied through the vacuum equalization passage.

In other embodiments, the bio-barrier membrane has sufficient deformability to at least substantially permit adherence to the device treatment surface in response to negative pressure in the device receiving chamber. The gasket member is configured in combination with the treatment device such that at least a substantial portion of the treatment surface is spaced about 1 mm from the bio-barrier membrane before application of a negative pressure and the bio-barrier membrane is plastically deformable in response to the negative pressure. The bio-barrier membrane is sufficiently deformable to deform about 1 mm over substantially its entire area without tearing. Also the bio-barrier membrane may comprise a polyethylene film with a thickness of about 0.0005 inches. The gas permeable membrane may comprise a hydrophobic film with a thickness of about 0.0005 inches.

In another exemplary embodiment of the applicator-tissue interface the bio-barrier membrane is configured and dimensioned to deform against the treatment surface with sufficient force to at least substantially eliminate bubbles between the treatment surface and bio-barrier membrane. The liquid and gas impermeable membrane may be selected of a material having sufficient strength to withstand a negative pressure of approximately −20 mm mercury +/−1 mm at a thickness of about 0.0005 inches

According to another embodiment, the applicator-tissue interface further comprises a downward depending resilient skirt surrounding the treatment opening and forming an extension of the tissue receiving chamber to facilitate acquisition of tissue within the tissue receiving chamber in response to negative pressure applied through the vacuum channel. The resilient skirt may be outwardly flared and may have at least one alignment marking disposed thereon.

In another embodiment, the applicator-tissue interface further comprises in combination a treatment template configured for adherence to a patient in a treatment area, wherein the treatment template includes a series of markers that when aligned with the alignment marking position the applicator-tissue interface in a proper treatment location for sequentially positioned treatments.

According to another embodiment of the invention a vacuum tube communicates with the vacuum channel through a port formed in the body member and a trap element formed in the vacuum tube to prevent outflow of materials received in the tissue receiving chamber through the vacuum tube.

In yet another embodiment of an applicator-tissue interface for use with a medical treatment device according to the present invention, the interface comprises a polycarbonate body member having a wall surrounding a treatment window and defining a tissue receiving chamber at a lower side and a device receiving chamber at an upper side; a polyethylene film having a thickness of about 0.0005 inches sealingly disposed across the treatment window between the tissue receiving chamber and the device receiving chamber to provide a bio-barrier there across, the bio-barrier being transparent to the treatment modality; a vacuum channel disposed in the tissue receiving chamber adjacent the bio-barrier and surrounding the treatment opening, the vacuum channel communicating with the tissue receiving chamber to provide a negative pressure therein; a vacuum equalization passage communicating between the vacuum channel and the device receiving chamber; and a hydrophobic film having a thickness of about 0.005 inches sealingly disposed across the vacuum equalization passage to prevent flow bodily fluids there through while permitting air to pass.

In further embodiments of the applicator-tissue interface a thermal plastic elastomeric skirt extends downward from the body member surrounding and further defining the tissue receiving chamber, wherein the elastomeric skirt comprises silicone and wherein the hydrophobic film comprises PTFE.

According to another embodiment of an applicator-tissue interface for use with a medical treatment device according to the invention, the interface comprises a body member having a forward end, a back end, an upper side configured and dimensioned to mate with the treatment device and a lower side adapted to engage tissue to be treated; first locking means disposed along the forward end and extending in an upward direction from the body member, the first locking means being configured and dimensioned to engage a locking element disposed on a forward end of the treatment device; second locking means formed in the back end and configured and dimensioned to engage at least two locking elements disposed on a back end of the treatment device; and a membrane extending across the body member separating the upper side from the lower side.

In further embodiments, the body member has an upper edge surrounding the upper side and the first locking means comprises a projection extending upward from the upper edge. The projection defines an opening configured and dimensioned to receive the forward end locking element on the treatment device and may further define a finger engageable protrusion for user manipulation. The second locking means comprises first and second spaced apart tabs formed in the body member back end. The first and second spaced apart tabs may terminate substantially at the body upper edge.

Alternatively, the first and second spaced apart tabs each define an opening configured and dimensioned to receive a back end locking element on the treatment device, wherein a tube is secured to the body member between the first and second spaced apart tabs.

In yet another embodiment, the applicator-tissue interface further comprises first and second guide protrusions formed inside the body member back end, the protrusions positioned on either side and outwardly with respect to the second locking means. The body member may be formed of a substantially rigid material.

A resilient gasket member may be secured inside the body member upper side, the gasket member being configured and dimensioned to matingly receive a treatment side of the treatment device in close proximity to the membrane. The membrane may comprise a bio-barrier impervious to bodily fluids. Such an embodiment may also a resilient skirt extending from the lower side and surrounding the bio-barrier membrane.

According to another embodiment of an applicator-tissue interface for use with a medical treatment device of the invention, the interface comprises a body member having a wall surrounding a treatment opening and defining a tissue receiving chamber at a lower side and a device receiving chamber at an upper side, the body member further having a forward end and a back end formed by the wall; first locking means disposed along the forward end and extending in an upward direction from the body member, the first locking means being configured and dimensioned to engage a locking element disposed on a forward end of the treatment device; second locking means formed in the back end and configured and dimensioned to engage at least two locking elements disposed on a back end of the treatment device; a liquid and gas impermeable membrane sealingly disposed across the treatment opening between the tissue receiving chamber and the device receiving chamber to provide a bio-barrier there across, the bio barrier being transparent to the treatment modality; a vacuum channel disposed in the tissue receiving chamber adjacent the bio-barrier and surrounding the treatment opening, the vacuum channel communicating with the tissue receiving chamber to provide a negative pressure therein; a vacuum equalization passage communicating between the vacuum channel and the device receiving chamber; and a liquid impermeable, gas permeable membrane sealingly disposed across the vacuum equalization passage to prevent flow bodily fluids there through.

According to a further embodiment of an applicator-tissue interface for use with a medical treatment device according to the invention, the interface comprises a body member having a wall surrounding a treatment window and defining a tissue receiving chamber at a lower side and a device receiving chamber at an upper side, the body member further having a forward end and a back end formed by the wall; a downward depending resilient skirt surrounding the treatment window and forming an extension of the tissue receiving; an alignment marking centered along at least one edge of the resilient skirt; first locking means disposed along the forward end and extending in an upward direction from the body member, the first locking means being configured and dimensioned to engage a locking element disposed on a forward end of the treatment device; second locking means formed in the back end and configured and dimensioned to engage at least two locking elements disposed on a back end of the treatment device; a liquid and gas impermeable membrane sealingly disposed across the treatment opening between the tissue receiving chamber and the device receiving chamber to provide a bio-barrier there across, the bio barrier being transparent to the treatment modality; a vacuum channel disposed in the tissue receiving chamber adjacent the bio-barrier and surrounding the treatment opening, the vacuum channel communicating with the tissue receiving chamber to provide a negative pressure therein; a vacuum equalization passage communicating between the vacuum channel and the device receiving chamber; a liquid impermeable, gas permeable membrane sealingly disposed across the vacuum equalization passage to prevent flow bodily fluids there through ;a vacuum tube communicating with the vacuum channel through a port formed in the body member; and a trap element formed in the vacuum tube to prevent outflow of materials received in the tissue receiving chamber through the vacuum tube. The resilient skirt may be outwardly flared.

Alternative the applicator-tissue interface may further comprises in combination a treatment template configured for adherence to a patient in a treatment area, wherein the treatment template includes a series of markers that when aligned with the alignment marking position the applicator-tissue interface in a proper treatment location for sequentially positioned treatments.

In yet another embodiment of the applicator-tissue interface the body member is formed from polycarbonate; the liquid and gas impermeable membrane comprises a polyethylene film having a thickness of about 0.0005 inches; a vacuum equalization passage communicating between the vacuum channel and the device receiving chamber; the gas permeable membrane comprises a hydrophobic film having a thickness of about 0.005; and the resilient skirt comprises a thermal plastic elastomeric material.

According to yet another embodiment of the invention, a method for delivering a treatment to tissue with a medical device through a applicator-tissue interface, wherein the applicator-tissue interface comprises a gas and liquid impermeable bio-barrier membrane defining a treatment window that is transparent to the treatment modality comprises placing the interface over the medical device, covering at least a treatment surface of the device; placing the medical device and interface with the bio-barrier membrane adjacent a tissue area to be treated; applying a negative pressure between the device treatment surface and the bio-barrier membrane; applying a negative pressure between the bio-barrier membrane and the tissue area to be treated to draw tissue into contact with the bio-barrier membrane; equalizing the negative pressure on opposite sides of the bio-barrier membrane; applying the treatment through the bio-barrier membrane; and ceasing treatment and removing the medical device and interface from adjacent the tissue area without drawing fluids into contact with the treatment surface.

In a further embodiment, the applying a negative pressure between the device treatment surface and the bio-barrier membrane displaces the bio-barrier membrane and contacts at least a portion of the bio-barrier membrane with the treatment surface. Alternatively, the applicator-tissue interface further comprises a body member surrounding the treatment window and defining a tissue receiving chamber at a lower side and a device receiving chamber at an upper side, the bio-barrier membrane dividing the chambers, and the negative pressure between the bio-barrier membrane and the tissue area to be treated is applied through a vacuum channel disposed with body member surrounding the treatment window.

In another embodiment of the method according to the invention, the negative pressure between the device treatment surface and the bio-barrier membrane is applied through a vacuum equalization passage communicating between the vacuum channel and the device receiving chamber. Alternatively, the applicator-tissue interface further comprises a gasket member disposed in the device receiving chamber around the treatment window; and the step of placing the interface over the medical device comprises sealingly receiving the device in the gasket member with the treatment surface facing the bio-barrier membrane.

According to a further embodiment of the invention, the applicator-tissue interface further comprises a downward depending resilient skirt surrounding the treatment window and forming an extension of the tissue receiving chamber; and the step of placing the medical device and interface comprises compressing the resilient skirt against tissue surrounding the tissue area to be treated and at least substantially sealingly engaging the tissue with the resilient skirt. Alternatively, the resilient skirt has at least one alignment marking disposed thereon; and the step of placing the medical device and interface further comprises positioning a treatment template over the tissue area to be treated and aligning the at least one alignment marking with the treatment template. Additionally, the step of applying treatment may comprise applying sequentially positioned overlapping treatments corresponding to locations on the treatment template.

In further alternative embodiments, the device receiving chamber may have dimensions of approximately 1.34 inches by approximately 0.63 inches so as to closely receive the applicator therein and the tissue receiving chamber may have dimensions of approximately 1.54 inches by approximately 0.7 inches with a depth including resilient skirt of approximately 6.5 mm to 11 mm.

Having described a limited number of embodiments of the present invention, it should be apparent to those of ordinary skill in the art that numerous other embodiments and modifications thereof are contemplated as falling within the scope of the present invention as defined by the appended claims.

What is claimed is:

1. An applicator-tissue interface for use with a medical treatment device, said interface comprising:
a body member having a wall surrounding a treatment opening and defining a tissue receiving chamber at a lower side and a device receiving chamber at an upper side;
a liquid and gas impermeable membrane sealingly disposed across said treatment opening between the tissue receiving chamber and the device receiving chamber to provide, a bio-barrier membrane there across, said bio barrier being transparent to the treatment modality;
a vacuum channel disposed in the tissue receiving chamber adjacent said bio-barrier and surrounding said treatment opening, said vacuum channel communicating with said tissue receiving chamber to provide a negative pressure therein;
a vacuum equalization passage communicating between said vacuum channel and said device receiving chamber; and
a liquid impermeable, gas permeable membrane sealingly disposed across said vacuum equalization passage to prevent flow bodily fluids there through.

2. The applicator-tissue interface of claim 1, wherein the vacuum channel defines at least one port adjacent the treatment opening communicating with the tissue receiving chamber.

3. The applicator-tissue interface of claim 2, wherein the vacuum channel defines plural ports distributed around the treatment opening.

4. The applicator-tissue interface of claim 1, wherein the vacuum channel defines a continuous slit opening adjacent the bio-barrier surrounding the treatment opening, said slit opening communicating with the tissue receiving chamber.

5. The applicator-tissue interface of claim 4, wherein said continuous slit defines at least one enlarged notch opening.

6. The applicator-tissue interface of claim 5, wherein said continuous slit defines plural notch openings distributed around the treatment opening.

7. The applicator-tissue interface of claim 1, wherein the vacuum equalization passage comprises a port communicating directly from the vacuum channel into the device receiving chamber.

8. The applicator-tissue interface of claim 7, wherein:
the body member defines an inwardly directed flange surrounding the treatment opening;
the liquid and gas impermeable bio-barrier membrane is sealing secured to said flange; and
the vacuum equalization passage comprises a hole through said flange with the liquid impermeable, gas permeable membrane sealed there across.

9. The applicator-tissue interface of claim 1, wherein:
the body member defines a vacuum inlet communicating with the vacuum channel;
vacuum equalization passage comprises a passage communicating between the vacuum inlet and the device receiving chamber; and
the liquid impermeable, gas permeable membrane is disposed in said passage.

10. The applicator-tissue interface of claim 1, further comprising a gasket member disposed in the device receiving chamber around the treatment opening, the gasket member being configured and dimensioned to sealingly receive the medical treatment device with a treatment surface thereof facing the bio-barrier membrane to facilitate formation of a vacuum between the device and the bio-barrier membrane when negative pressure is applied through said vacuum equalization passage.

11. The applicator-tissue interface of claim 10, wherein the bio-barrier membrane has sufficient deformability to at least substantially permit adherence to the device treatment surface in response to negative pressure in the device receiving chamber.

12. The applicator-tissue interface of claim 11, wherein in said gasket member is configured in combination with the treatment device such that at least a substantial portion of the treatment surface is spaced about 1 mm from the bio-barrier membrane before application of a negative pressure.

13. The applicator-tissue interface of claim 11, wherein the bio-barrier membrane is plastically deformable in response to the negative pressure.

14. The applicator-tissue interface of claim 13, wherein the bio-barrier membrane is sufficiently deformable to deform about 1 mm over substantially its entire area without tearing

15. The applicator-tissue interface of claim 13, wherein the bio-barrier membrane comprises a polyethylene film with a thickness of about 0.0005 inches.

16. The applicator-tissue interface of claim 15, wherein the gas permeable membrane comprises a hydrophobic film with a thickness of about 0.0005 inches.

17. The applicator-tissue interface of claim 13, wherein the bio-barrier membrane deforms is configured and dimensioned to deform against the treatment surface with sufficient force to at least substantially eliminate bubbles between the treatment surface and bio-barrier membrane.

18. The applicator-tissue interface of claim 13, wherein the liquid and gas impermeable membrane is selected of a material having sufficient strength to withstand a negative pressure of approximately −20 mm mercury +/− 1 mm at a thickness of about 0.0005 inches

19. The applicator-tissue interface of claim 1, further comprising a downward depending resilient skirt surrounding the treatment opening and forming an extension of the tissue receiving chamber to facilitate acquisition of tissue within the tissue receiving chamber in response to negative pressure applied through the vacuum channel.

20. The applicator-tissue interface of claim 19, wherein the resilient skirt is outwardly flared.

21. The applicator-tissue interface of claim 20, wherein the resilient skirt has at least one alignment marking disposed thereon.

22. The applicator-tissue interface of claim 21, further comprising in combination a treatment template configured for adherence to a patient in a treatment area, wherein the treatment template includes a series of markers that when aligned with said alignment marking position the applicator-tissue interface in a proper treatment location for sequentially positioned treatments.

23. The applicator-tissue interface of claim 1, further comprising a vacuum tube communicating with the vacuum channel through a port formed in the body member and a trap element formed in the vacuum tube to prevent outflow of materials received in the tissue receiving chamber through said vacuum tube.

24. An applicator-tissue interface for use with a medical treatment device, said interface comprising:
a polycarbonate body member having a wall surrounding a treatment window and defining a tissue receiving chamber at a lower side and a device receiving chamber at an upper side;
a polyethylene film having a thickness of about 0.0005 inches sealingly disposed across said treatment window between the tissue receiving chamber and the device receiving chamber to provide a bio-barrier there across, said bio-barrier being transparent to the treatment modality;
a vacuum channel disposed in the tissue receiving chamber adjacent said bio-barrier and surrounding said treatment opening, said vacuum channel communicating with said tissue receiving chamber to provide a negative pressure therein;
a vacuum equalization passage communicating between said vacuum channel and said device receiving chamber; and
a hydrophobic film having a thickness of about 0.005 inches sealingly disposed across said vacuum equalization passage to prevent flow bodily fluids there through while permitting air to pass.

25. The applicator-tissue interface of claim 24, further comprising a thermal plastic elastomeric skirt extending downward from the body member surrounding and further defining the tissue receiving chamber.

26. The applicator-tissue interface of claim 25, wherein the elastomeric skirt comprises silicone.

27. The applicator-tissue interface of claim 20, wherein the hydrophobic film comprises PTFE.

28. An applicator-tissue interface for use with a medical treatment device, said interface comprising:
a body member having a forward end, a back end, an upper side configured and dimensioned to mate with the treatment device and a lower side adapted to engage tissue to be treated;
first locking means disposed along said forward end and extending in an upward direction from the body member, said first locking means being configured and dimensioned to engage a locking element disposed on a forward end of the treatment device;
second locking means formed in said back end and configured and dimensioned to engage at least two locking elements disposed on a back end of the treatment device; and
a membrane extending across the body member separating the upper side from the lower side.

29. The applicator-tissue interface of claim 28, wherein said body member has an upper edge surrounding the upper side and said first locking means comprises a projection extending upward from said upper edge.

30. The applicator-tissue interface of claim 29, wherein said projection defines an opening configured and dimensioned to receive the forward end locking element on the treatment device.

31. The applicator-tissue interface of claim 30, wherein said projection further defines a finger engageable protrusion for user manipulation.

32. The applicator-tissue interface of claim 29, wherein said second locking means comprises first and second spaced apart tabs formed in said body member back end.

33. The applicator-tissue interface of claim 32, wherein said first and second spaced apart tabs terminate substantially at said body upper edge.

34. The applicator-tissue interface of claim 33, wherein said first and second spaced apart tabs each define an opening configured and dimensioned to receive a back end locking element on the treatment device.

35. The applicator-tissue interface of claim 32, wherein a tube is secured to the body member between said first and second spaced apart tabs.

36. The applicator-tissue interface of claim 28, further comprising first and second guide protrusions formed inside the body member back end, said protrusions positioned on either side and outwardly with respect to said second locking means.

37. The applicator-tissue interface of claim 28, wherein said body member is formed of a substantially rigid material.

38. The applicator-tissue interface of claim 37, further comprising a resilient gasket member secured inside the body member upper side, said gasket member being configured and dimensioned to matingly receive a treatment side of the treatment device in close proximity to said membrane.

39. The disposable of claim 38, wherein said membrane comprises a bio-barrier impervious to bodily fluids.

40. The disposable of claim 39, further comprising a resilient skirt extending from said lower side and surrounding the bio-barrier membrane.

41. An applicator-tissue interface for use with a medical treatment device, said interface comprising:
a body member having a wall surrounding a treatment opening and defining a tissue receiving chamber at a lower side and a device receiving chamber at an upper side, the body member further having a forward end and a back end formed by said wall;
first locking means disposed along said forward end and extending in an upward direction from the body member, said first locking means being configured and dimensioned to engage a locking element disposed on a forward end of the treatment device;
second locking means formed in said back end and configured and dimensioned to engage at least two locking elements disposed on a back end of the treatment device;
a liquid and gas impermeable membrane sealingly disposed across said treatment opening between the tissue receiving chamber and the device receiving chamber to provide a bio-barrier there across, said bio barrier being transparent to the treatment modality;
a vacuum channel disposed in the tissue receiving chamber adjacent said bio-barrier and surrounding said treatment opening, said vacuum channel communicating with said tissue receiving chamber to provide a negative pressure therein;
a vacuum equalization passage communicating between said vacuum channel and said device receiving chamber; and
a liquid impermeable, gas permeable membrane sealingly disposed across said vacuum equalization passage to prevent flow bodily fluids there through.

42. An applicator-tissue interface for use with a medical treatment device, said interface comprising:
a body member having a wall surrounding a treatment window and defining a tissue receiving chamber at a lower side and a device receiving chamber at an upper side, the body member further having a forward end and a back end formed by said wall;
a downward depending resilient skirt surrounding the treatment window and forming an extension of the tissue receiving;
an alignment marking centered along at least one edge of the resilient sikt;
first locking means disposed along said forward end and extending in an upward direction from the body member, said first locking means being configured and dimensioned to engage a locking element disposed on a forward end of the treatment device;
second locking means formed in said back end and configured and dimensioned to engage at least two locking elements disposed on a back end of the treatment device;
a liquid and gas impermeable membrane sealingly disposed across said treatment opening between the tissue receiving chamber and the device receiving chamber to provide a bio-barrier there across, said bio barrier being transparent to the treatment modality;
a vacuum channel disposed in the tissue receiving chamber adjacent said bio-barrier and surrounding said treatment opening, said vacuum channel communicating with said tissue receiving chamber to provide a negative pressure therein;
a vacuum equalization passage communicating between said vacuum channel and said device receiving chamber;
a liquid impermeable, gas permeable membrane sealingly disposed across said vacuum equalization passage to prevent flow bodily fluids there through;
a vacuum tube communicating with the vacuum channel through a port formed in the body member; and
a trap element formed in the vacuum tube to prevent outflow of materials received in the tissue receiving chamber through said vacuum tube

43. The applicator-tissue interface of claim 42, wherein the resilient skirt is outwardly flared.

44. The applicator-tissue interface of claim 42, further comprising in combination a treatment template configured for adherence to a patient in a treatment area, wherein the treatment template includes a series of markers that when aligned with said alignment marking position the applicator-tissue interface in a proper treatment location for sequentially positioned treatments.

45. The applicator-tissue interface of claim 42, wherein:
the body member is formed from polycarbonate;
the liquid and gas impermeable membrane comprises a polyethylene film having a thickness of about 0.0005 inches;
a vacuum equalization passage communicating between said vacuum channel and said device receiving chamber;
the gas permeable membrane comprises a hydrophobic film having a thickness of about 0.005; and
the resilient skirt comprises a thermal plastic elastomeric material.

46. A method for delivering a treatment to tissue with a medical device through a applicator-tissue interface, wherein the applicator-tissue interface comprises a gas and liquid impermeable bio-barrier membrane defining a treatment window that is transparent to the treatment modality, said method comprising:
placing the interface over the medical device, covering at least a treatment surface of the device;
placing the medical device and interface with the bio-barrier membrane adjacent a tissue area to be treated;
applying a negative pressure between the device treatment surface and the bio-barrier membrane;
applying a negative pressure between the bio-barrier membrane and the tissue area to be treated to draw tissue into contact with the bio-barrier membrane;
equalizing the negative pressure on opposite sides of the bio-barrier membrane;
applying the treatment through the bio-barrier membrane; and
ceasing treatment and removing the medical device and interface from adjacent the tissue area without drawing fluids into contact with the treatment surface.

47. The method of claim 46, wherein said applying a negative pressure between the device treatment surface and the bio-barrier membrane displaces the bio-barrier membrane and contacts at least a portion of the bio-barrier membrane with the treatment surface.

48. The method of claim 47, wherein:
the applicator-tissue interface further comprises a body member surrounding the treatment window and defining a tissue receiving chamber at a lower side and a device receiving chamber at an upper side, the bio-barrier membrane dividing said chambers, and
the negative pressure between the bio-barrier membrane and the tissue area to be treated is applied through a vacuum channel disposed with body member surrounding the treatment window.

49. The method of claim 48, wherein said negative pressure between the device treatment surface and the bio-barrier membrane is applied through a vacuum equalization passage communicating between the vacuum channel and the device receiving chamber.

50. The method of claim 49, wherein:
the applicator-tissue interface further comprises a gasket member disposed in the device receiving chamber around the treatment window; and
said step of placing the interface over the medical device comprises sealingly receiving the said device in said gasket member with the treatment surface facing the bio-barrier membrane.

51. The method of claim 50, wherein:
the applicator-tissue interface further comprises a downward depending resilient skirt surrounding the treatment window and forming an extension of the tissue receiving chamber; and
said step of placing the medical device and interface comprises compressing the resilient skirt against tissue surrounding the tissue area to be treated and at least substantially sealingly engaging said tissue with said resilient skirt.

52. The method of claim 51, wherein:
the resilient skirt has at least one alignment marking disposed thereon; and
said step of placing the medical device and interface further comprises positioning a treatment template over the tissue area to be treated and aligning the at least one alignment marking with the treatment template.

53. The method of claim 52, wherein said step of applying treatment comprises applying sequentially positioned overlapping treatments corresponding to locations on the treatment template.

54. The applicator-tissue interface of claim 1, wherein the device receiving chamber has dimensions of approximately 1.34 inches by approximately 0.63 inches so as to closely receive the applicator therein.

55. The applicator-tissue interface of claim 1, wherein the tissue receiving chamber has dimensions of approximately 1.54 inches by approximately 0.7 inches with a depth including resilient skirt of approximately 6.5 mm to 11 mm.
